(19) United States
(12) Patent Application Publication
(10) Pub. No.: US 2018/0333542 A1
(43) Pub. Date: Nov. 22, 2018

(54) JOINT ASPIRATION DEVICE

(71) Applicant: Sean Fitzsimmons, New York, NY (US)

(72) Inventor: Sean Fitzsimmons, New York, NY (US)

(21) Appl. No.: 15/985,940

(22) Filed: May 22, 2018

Related U.S. Application Data
(60) Provisional application No. 62/509,265, filed on May 22, 2017.

(51) Int. Cl. A61M 5/32; A61B 17/34

(52) U.S. Cl. CPC A61M 5/329; A61M 5/343; A61B 17/3417

(57) ABSTRACT

A joint aspiration device having a hub, a needle, and a platform. The hub is adapted to engage with an external syringe. The platform is formed over a portion of the needle adjacent to the hub. The platform has a textured surface to enhance leverage and rotational control when engaging and disengaging the joint aspiration device from an external syringe.

### CROSS-REFERENCE TO RELATED APPLICATIONS

This Non-Provisional Patent Application claims the benefit of the filing date of U.S. Provisional Patent Application Ser. No. 62/509,265, filed May 22, 2017, the entire teachings of which are incorporated herein by reference.

### BACKGROUND

The present disclosure relates to devices for aspirating fluid from, or injecting fluid into, a human body. More particularly, the present disclosure relates to needle-based devices for aspirating fluid from, or injecting fluid into, a human joint.

Joint aspiration is a medical procedure in which a practitioner drains fluid from a patient's joint. After aspiration, the practitioner often injects fluid into the joint utilizing a sterile needle connected to a syringe. Fluid from the joint can be tested and analyzed to determine the cause of joint swelling. Joint aspiration can be used to diagnose trauma, gout, arthritis, infections of the joint, etc. Additionally, removal of excess fluid from the joint can reduce swelling of the joint and decrease pain. Fluids injected into the joint during a joint aspiration procedure can include corticosteroids or local anesthetics. Most commonly, the joint aspiration procedure is performed on the knee joint.

During a joint aspiration procedure, a practitioner inserts a needle, which is attached to an external syringe, though the skin into the joint. Once inserted into the joint, a plunger of the syringe can be pulled backwards to create a vacuum inside a body of the syringe. The suction of the vacuum pulls excess fluid from the joint into the body of the syringe. The filled syringe may then be disengaged from the needle, while keeping the needle inserted into the joint. A second pre-filled medication syringe may then be engaged with the needle. The fluid from the syringe may be injected into the joint by pressing the plunger of the syringe forward. Once aspiration and/or injection is complete, the needle may be withdrawn from the joint, and pressure may be applied with sterile gauze, and an adhesive bandage applied to the needle's entry point. In some situations, the volume of fluid in the joint is greater than the capacity of the syringe used and a now-filled syringe needs to be disengaged and a new, empty syringe is attached to the needle to complete the fluid removal procedure.

A common problem with previous joint aspiration devices is controlled handling thereof by the physician. For example, many available joint aspiration devices are not conducive to practitioner grasping while at the same time providing rotational control of the needle during engagement or disengagement from the syringe. Another potential concern is slippage occurring when a practitioner engages or disengages a syringe from the device. This slippage can causes the practitioner's fingers to slide from the non-sterile portion of the hub of the device to the sterile portion of the device, thus causing a breach in sterility. This can increase the patient's risk of infection. Furthermore, there is a risk of exposure to bodily fluids by the practitioner if slippage occurs during engagement or disengagement of the joint aspiration needle from the syringe. Previous joint aspiration devices do not provide a sufficient surface for the practitioner to grasp and control while rotating the syringe during engagement or disengagement from the needle. Prior art devices do not have structures that may be easily gripped and simultaneously provide rotational control of the device by the physician during syringe engagement or disengagement. Moreover, some prior art devices require the practitioner to grasp the hub when engaging or disengaging the device from a syringe, which increases the chance of breaching sterility.

Therefore, there is a need to provide a device that provides rotational control and leverage when engaging and disengaging the needle and the hub from an external syringe, thus limiting the risk of a breach of sterility.

### SUMMARY

Some aspects the present disclosure relate to a useful alternative to existing joint aspiration devices that fail to provide rotational control and leverage when engaging and disengaging a joint aspiration device from an external syringe. In one embodiment, the joint aspiration device may include a hub, a needle, and a platform. The joint aspiration device may include a platform formed (e.g., molded) over a segment of the needle; the so-encompassed segment can be referred to as a cannula portion of the needle. The remaining segment of the needle (i.e., outside of or beyond the platform) can be referred to as an exposed needle portion. The joint aspiration device may have a continuous lumen extending from the hub to a distal tip of the needle. The joint aspiration device has a proximal end and a distal end. At the proximal end of the joint aspiration device, the hub may be configured to engage with an external syringe through various standard types of connections, including a luer lock. At the distal end of the joint aspiration device, the needle may have a bevel tip for insertion into a joint.

Other aspects of the present disclosure provide a joint aspiration devices that can include a platform for a practitioner to grasp during engagement and disengagement of the joint aspiration device from an external syringe. The platform may be disposed between the hub and the exposed needle portion. In some embodiments, the platform is offset from the hub in order to decrease the possibility of the practitioner's fingers slipping from the platform to the hub during syringe engagement and disengagement. The platform may be disc shaped. The platform may define opposing edges extending from opposite sides of the needle. One or both of the edges can incorporate a curvature (e.g., convex curve) in extension from the needle. In other embodiments, the platform may be generally square shaped. At least one major surface of the platform surface may be textured to enhance leverage and rotational control by a practitioner when grasping the platform during syringe engagement or disengagement.

### BRIEF DESCRIPTION OF THE DRAWINGS

FIG. 1 is a perspective view of a joint aspiration device in accordance with principles of the present disclosure.

FIG. 2 is a top view of the joint aspiration device of FIG. 1.

FIG. 3 is a side view of the joint aspiration device of FIG. 1.

FIG. 4 is a cross sectional view of the joint aspiration device of FIG. 1.

### DETAILED DESCRIPTION

The following disclosure describes embodiments of a joint aspiration device including a hub, a needle, and a platform that provides for rotational control and leverage of the device when engaging and disengaging from an external syringe.

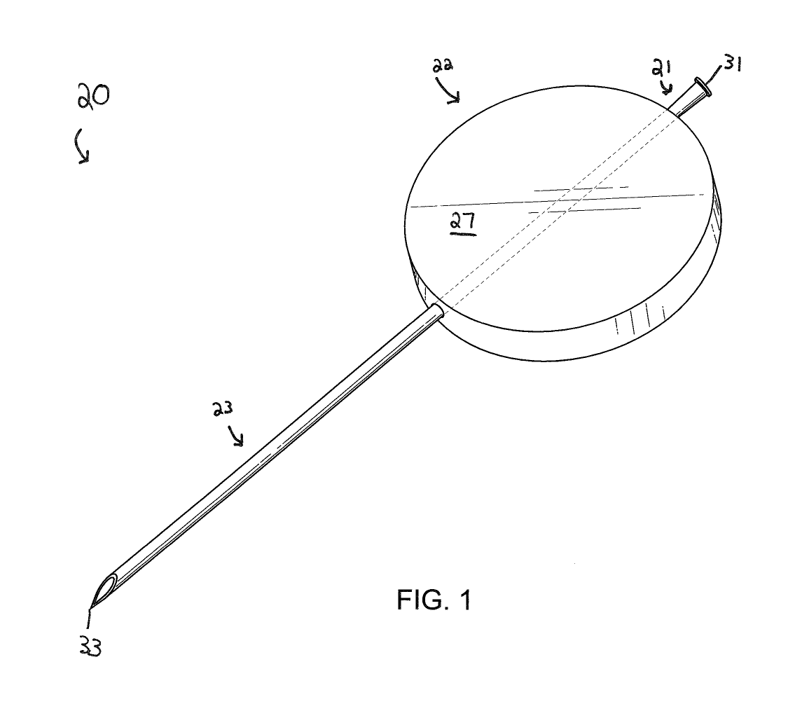

FIG. 1 illustrates one embodiment of a joint aspiration device 20 in accordance with principles of the present disclosure. The joint aspiration device 20 shown includes a hub 21, a needle 23, and a platform 22. The hub 21 is located at a proximal end 31 of the joint aspiration device 20. The needle 23 extends from the hub 21 to a distal end 33 of the joint aspiration device 20.

The hub 21 is configured to engage with an external syringe (not shown). The engagement configuration of the hub 21 with the external syringe may incorporate a luer lock, or may be a screw-type configuration or any other configuration that is common in the field.

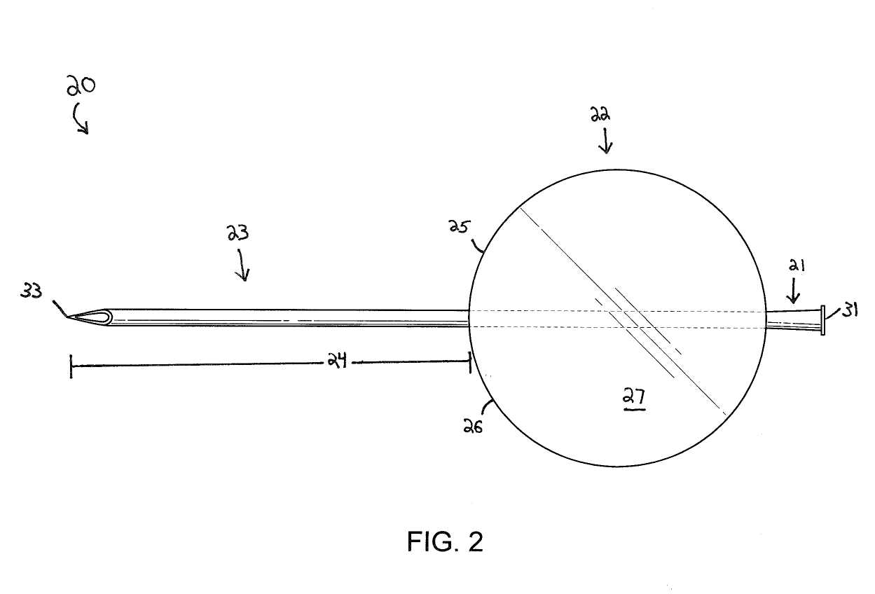

As illustrated in FIG. 2, the needle 23 can be viewed as defining an exposed portion 24 extending distally from the platform 22 to the distal end 33 of the joint aspiration device 20. The exposed needle portion 24 can be of different and varying gauges and sizes to accommodate different sized individuals or different joint sizes. For example, the exposed needle portion 24 may be an 18 gauge, 1.5 inch needle. In some embodiments, the exposed needle portion 24 can be in the 12-27 gauge range and has a length ranging from 2.54 cm to 8.89 cm (1 inch to 3.5 inches). Other dimensions are also envisioned. The exposed needle portion 24 may terminate at a beveled or sharpened tip.

The platform 22 is formed over the needle 23 and can be a plastic material molded onto the needle 23 adjacent to the hub 21. A portion of the needle 23 thus extends through the platform 22 and to define a cannula portion 34. The platform 22 may be a solid material molded around the cannula portion 34 or may be a hollow shell molded around the cannula portion 34. The platform 22 can have various shapes and sizes. In some embodiments, a shape of the platform 22 can be symmetrical relative to a central axis defined by the needle 23; in other embodiments, a non-symmetrical shape or arrangement can be provided. Regardless, projection of the platform 22 relative to the needle 23 can be viewed as defining first and second opposing edges 25, 26, and first and second opposing major surfaces 27, 28. The edges 25, 26 extend from opposite sides of the needle 23, respectively, and in some embodiments can have an arcuate shape, such as the convex curvature shown. Other shapes or geometry features can be incorporated into one or both of the edges 25, 26, such as one more curves (convex or concave), linear segments, etc. The opposing major surfaces 27, 28 can have an identical shape and size as shown, or alternatively can exhibit one or more differences in geometry. For example, one or both of the major surfaces 27, 28 can have the circular or disc-like shape as shown. To provide rotational control and leverage of the joint aspiration device 20 when a practitioner grasps the platform 22 during engagement or disengagement of the device from an external syringe, one or both of the major surfaces 27, 28 may have various textures. In one embodiment, one or both of the major surface 27, 28 may be textured with raised bumps, indents, surface coating with high coefficient of friction, etc.

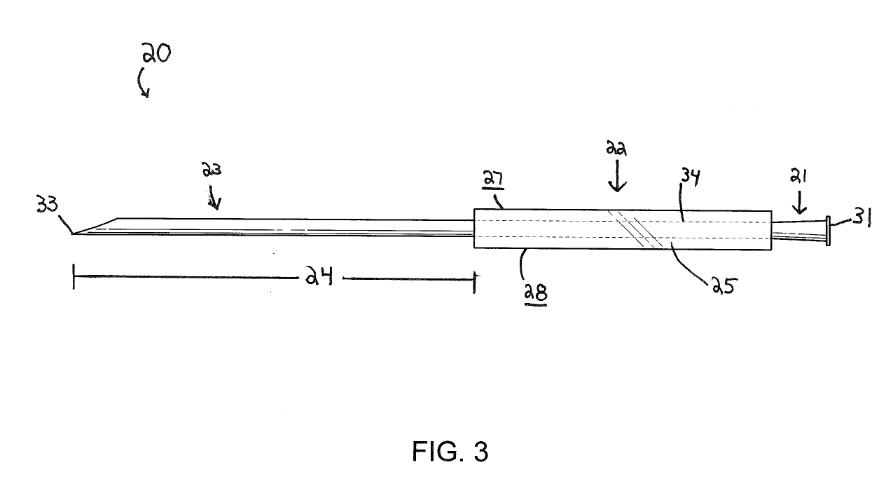

As described above, the platform 22 can have a generally disc shape as shown in FIG. 2. In other embodiments, the platform 22 may form other shapes, for example the platform 22 may have a generally square shape. The platform 22, having a disc shape, may have a diameter in the range of 5 mm-50 mm, alternatively in the range of 20 mm-35 mm, and in other embodiments in the range of about 25 mm to 28 mm. Where the platform 22 has other shapes, a corresponding major outer dimension thereof can also be in accordance with these ranges. Other dimensions, greater or lesser, are also acceptable. As shown in FIG. 3, the platform 22 has a thickness (i.e., linear distance between the opposing major surfaces 27, 28) that is greater than an outer diameter of the cannula portion 34. In some non-limiting embodiments, the platform 22 may have a thickness in the range of 1.0-50.0 mm, alternatively in the range of 2.0-6.0 mm, and in other embodiments in the range of about 1.5 mm to 3.5 mm. Other thickness dimensions are also acceptable. Along these same lines, while the opposing major surfaces 27, 28 are illustrated as being substantially flat (i.e., within 10% of truly flat surface) and substantially parallel (i.e., within 10% of a truly parallel relationship), in other embodiments, one or both of the major surfaces 27, 28 can exhibit or incorporate surface features or geometries that deviate from being substantially flat and/or substantially parallel.

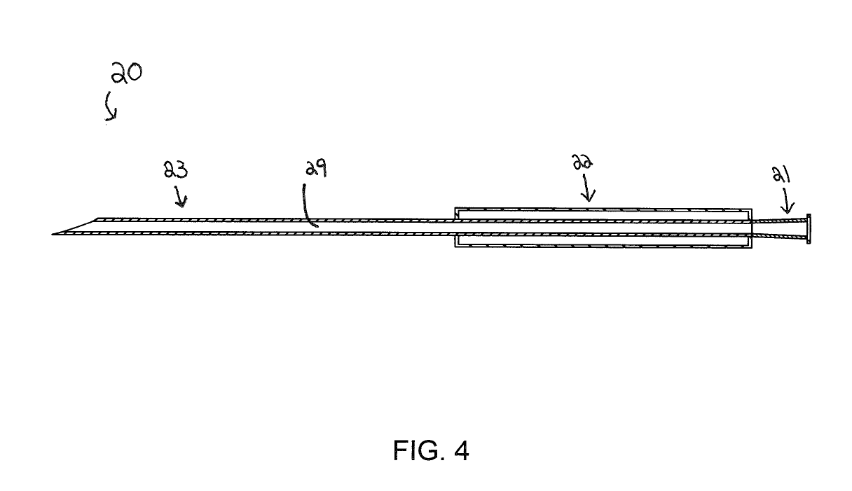

FIG. 4 illustrates a cross sectional view of the joint aspiration device 20 and shows that a continuous lumen 29 extends from the proximal end 31 to the distal end 33 through the hub 21 and the needle 23, including through the cannula portion 34 and the exposed needle portion 24. As a point of reference, the needle 23 can be akin to a conventional surgical needle useful for joint aspiration procedures, such as a hollow metal tube configured and used for aspiration of fluid into an external syringe and injection of fluid from an external syringe.

Although the present disclosure has been described with reference to preferred embodiments, workers skilled in the art will recognize that changes can be made in form and detail without departing from the spirit and scope of the present disclosure.

What is claimed is:

1. A joint aspiration device comprising:
a hub adapted to engage with an external syringe;
a needle extending from the hub; and
a platform formed over the needle, wherein the platform is adapted to be manually grasped during external syringe engagement or disengagement.

2. The joint aspiration device of claim 1, wherein at least one major surface of the platform is textured

3. The joint aspiration device of claim 1, wherein a continuous lumen extends from the hub to a distal tip of the needle.

4. The joint aspiration device of claim 1, wherein the platform defines opposing, first and second edges extending from opposite sides of the needle.

5. The joint aspiration device of claim 4, wherein at least one of the first and second edges is round.

6. The joint aspiration device of claim 4, wherein at least one of the first and second edges defines a convex curve.

7. The joint aspiration device of claim 1, wherein the platform defines a major outer dimension in the range of 20-35 mm.

8. The joint aspiration device of claim 1, wherein the platform defines a length in a direction parallel with an central axis of the needle, a width in a direction perpendicular to the length, and a thickness in a direction perpendicular to the width and the length, and further wherein the length and the width are greater than the thickness, and even further wherein the thickness is greater than a diameter of the needle.

9. The joint aspiration device of claim 8, wherein the thickness is in the range of

2.0-6.0 mm.

10. The joint aspiration device of claim 1, wherein the platform defines opposing major surfaces, and further wherein each of the opposing major surfaces is substantially flat.

11. The joint aspiration device of claim 1, wherein the platform is a plastic material molded over the needle.

12. The joint aspiration device of claim 1, wherein an exposed portion of the needle projects from an edge of the platform to a needle tip, and further wherein the exposed portion has a length in the range of 2.54-8.89 cm.

13. The joint aspiration device of claim 1, wherein the hub includes a luer lock.

14. A method for treating a joint of a patient, the method comprising:
grasping a platform of a joint aspiration device in a first hand of a user, the joint aspiration device further including a needle extending from the platform and a hub;
attaching a first syringe held in a second hand of the user to the hub while continuing to grasp the platform with the first hand;
inserting the needle into the joint while continuing to grasp the platform with the first hand; and
operating the first syringe to extract fluid from the joint through the needle while continuing to grasp the platform with the first hand.

15. The method of claim 14, further comprising:
rotating the first syringe relative to the joint aspiration device to disengage the first syringe from the hub while continuing to grasp the platform with the first hand and maintaining the needle in the joint.

16. The method of claim 15, further comprising:
attaching a second syringe to the hub, including rotating the second syringe relative to the joint aspiration device, while continuing to grasp the platform with the first hand and maintaining the needle in the joint.

17. The method of claim 16, further comprising:
operating the second syringe to inject fluid into the joint via the needle while continuing to grasp the platform with the first hand.
